(19) United States
(12) Patent Application Publication
(10) Pub. No.: US 2011/0224482 A1
(43) Pub. Date: Sep. 15, 2011

(54) Intimate Inseminator

(75) Inventors: Stefanie A. McCarthy, San Diego, CA, CA (US); Taneashia R. Morrell, San Ysidro, CA, CA (US); Tamara Burton, San Diego, CA (US)

(73) Assignees: Stefanie Alicia McCarthy, San Diego, CA (US); Taneashia Renee Morrell, San Ysidro, CA (US)

(21) Appl. No.: 12/721,094

(22) Filed: Mar. 10, 2010

(51) Int. Cl. A61B 17/43

(52) U.S. Cl. 600/35

(57) ABSTRACT

A sexual device to be worn externally by a woman, man, or transgender person, that will allow said wearer to artificially inseminate a sexual partner by use of pseudo natural means. That is, a harness device constructed material so that the frontal lobe of the device is a dildo with cylindrical tubing constructed to run horizontally through the center, with a predetermined length of tubing remaining outside the distal end of dildo; the distal end of tubing is attached to a syringe that is attached to the side of the harness so that the wearer can manipulate the amount of semen fluid that flows through the syringe. The device provides a method for pseudo genital-to-genital sexual contact between two partners to artificially inseminate in a way other than the use of the “syringe through the vaginal canal” method, or any other methods that are currently used for artificial insemination.

### BACKGROUND OF THE INVENTION

This invention concerns methods for sexual partners to artificially inseminate by using a sexual harness device that allows for pseudo genital-to-genital contact between sexual partners.

Preferred embodiment harness (FIGS. 1, 7, 8) as shown in FIG. 1, Intimate Inseminator of the preferred embodiment of the invention comprises a harness having an opening for a dildo (1P), rounded straps affixed firmly to hold a 5 ml syringe (1D, 1E, 1F, 1G), leg straps (1B, 1C), leg strap buckles (1L, 1N), a center strap which begins at the bottom of the flange and continues to the rear of the center waist strap (1H), flange (1A), female snap fasteners (1Q). The total infrastructure of the Intimate Inseminator is designed to fit all body types.

As shown in FIGS. 7 and 8, the head of the flange (7A, 8C) has an elongated member (dildo) (7E, 8B), which base is attached to the female snap fasteners (7F, 8D), the dildo has a cylindrical tube (7K, 8F) running horizontally through the dildo, beginning at the head of the dildo, continuing through its core, exiting through its end and continuing on for a predetermined length, attaching to the orifice (head) of a 5 ml syringe (7I, 8J).

As shown in FIG. 2, the dildo (2A) is attached to a rubber backing (2F) with male fasteners (2G) to fit into the flange's female fasteners (1Q, 7F, 8D), testicles (2C, 2D) are incorporated into the dildo to give it a more natural feel.

As shown in FIGS. 3 and 4, the dildo (3B, 4D) is attached to a rubber backing (3H, 4H) with male fasteners (3I, 4I) to fit into the flange's female fasteners (1Q, 7F, 8D), testicles (3E, 3F, 4E, 4F) cylindrical tubing (3C, 4B) with a predetermined length and width running horizontally through the core of the dildo exiting through its end and continuing on for a predetermined length (3D, 4C) so that its distal end can be attached to a 5 ml syringe (FIG. 6).

As shown in FIG. 5, cylindrical tube to be used to transport material fluid whereas both ends of said tube being open. 5B is placed at the head of the dildo (3A, 4A), and the distal end (5C) is connected to a 5 ml syringe (FIG. 6).

As shown in FIG. 6, the syringe is a simple piston pump consisting of a plunger (6D) that fits together in a tube (barrel, 6C). The orifice (opening)(6A) at the head of the syringe allows liquid to enter or exit through the barrel. The rubber piston (6F) is connected to the plunger and helps push/pull liquid through the orifice. The barrel (6C) is made of plastic or glass and houses the plunger and liquid that is contained in its chamber. The plunger (6D) pushes/pulls liquid through the barrel. The plunger tip (6E) allows the user to push/pull liquid through the orifice. Graduated marks (6B) indicate the volume of fluid inside the barrel.

As shown in FIGS. 9 and 10, the sheath (9B, 10B) is a close fitting covering or case for the purpose of holding an object. That object in this devise would be a penis (9A, 10A). The sheath will have cylindrical tubing (9C, 10C) running trough its center core and exiting through its end to be attached to a 5 ml syringe (FIG. 6). Male snap fasteners (9D, 10D) are constructed at the bottom end of the sheath to be attached to the female snap fasteners (1Q, 7F, 8D) of the flange (1A, 7A, 8C).

### SUMMARY, HISTORY AND BACKGROUND OF INVENTION

Modern technology has allowed sexual partners to artificially inseminate by placing a syringe in the vaginal canal and manually injecting sperm through the syringe and into the sub cavity of a vagina. Two female partners and transgender partners have been using syringe injection methods for a number of years. Additionally, heterosexual couples experiencing impotency, low sperm count, testicular cancer issues or the like, have also used these methods. The syringe method allows each subgroup mentioned above to impregnate their partners, by using a conventional method. Although using the manual syringe method has shown to be successful, it is also impersonal.

By allowing a sexual partner to wear a sexual device comprised of a harness with a dildo (as a penis substitute) attached to the frontal lobe of the harness, having a cylindrical tube running through the inside of said dildo, and being attached at the end to a syringe filled with semen, the sexual partner wearing said device is able to manually inseminate their partner while actually having pseudo genital-to-genital sexual intercourse; thereby allowing the partners to have an intimate sexual experience during the artificial insemination process.

### Harness:

Two straps, three straps and corset harnesses are quite common. The first part of a strap on setup is the harness, which connects the dildo to the wearer's body, usually in a position similar to that of a male's genitals. A good harness should be sturdy yet comfortable, and is often designed to provide stimulation for the wearer.

a) Leather material is comfortable and adjusts to the wearer's body but still is strong

b) Metal is an element, compound, or alloy material. The synthetic materials often have the characteristic of silvery-grey reflectiveness (luster) of elemental materials. Some metals and metal alloys possess high structural strength per unit mass, making them useful materials for carrying large loads or resisting impact damage. The strength and resilience of metals has led to their frequent use in sexual harnesses. There are shiny nickel-plate, sinister gun metal and gold-tone gilt finish as well as stripped, dual-tone combination metal harnesses.

c) Lacy material is an openwork fabric, patterned with open holes in the work, made by machine or hand. The holes can be formed by looping a thread, twisting or braiding it to the other threads independently from a backing fabric.

d) Cloth material is used mostly for clothing extremely inexpensive harnesses, which are often rendered as uncomfortable and useless. However, some sexual partners prefer the feel of cloth material, to that of any other material.

e) Nylon webbing is relatively affordable and very durable. Nylon material is easy to clean and requires little maintenance.

f) Some harnesses are made with soft plastic, such as flexible vinyl. These are often available in unique colors, such as completely transparent, which is not possible with other materials.

g) Latex harnesses usually cover the entire area, rather than just using straps. They usually have a limited lifespan, and require much care (e.g., special cleaners or shiners to keep the latex from turning dry or getting dusty).

h) Environmentally friendly harnesses are created to inflict minimal or no harm to the environment. The green movement is making huge stride replacing toxins and waste in the marketplace with organic fibers like: bamboo, cork, teak, corn starch, hemp, soybean, organic cotton, recycled glass, recycled polyester felt, aluminum and rubber.

### Dildo:

A dildo is a sexual device resembling a penis in shape, size and overall appearance. These devices are often used by people of all genders and sexual orientations. There are dildos designed to be worn in a harness (sometimes called a strap-on dildo), or to be worn inside, sometimes with vibrating devices attached externally.

Strap on dildos may be double-ended, in which case they are meant to be worn by users who want to experience vaginal or anal penetration while also penetrating a partner. Other types of dildos include those designed to be fitted to the face of one party, inflammable dildos, and dildos with suction cups attached to the base. There are also thigh, face and furniture mounting straps.

A strap on dildo is designed to be worked with a harness during sexual activity, harnesses and dildos are made in a wide variety of styles, with variations in how the harness fits the wearer, how the dildo attaches to the harness, as well as various features intended to facilitate stimulation of the wearer or sexual partner.

Strap on dildos can be used by people of any sex or gender. A person who does not have a penis may use a strap on, as may a person whose penis does not allow for penetration, whether due to size or function (e.g., a transgender, impotent men, lesbian partners, or the like.).

Hollowed dildos (sheaths) are often sold as penis extensions, but the most common use is for men with erectile dysfunction. The penis is inserted into the hollow inside of the dildo, then the harness is put on, allowing the man to penetrate his partner with the dildo.

### Cylindrical Tubing:

An elongated tubular body formed of synthetic plastic material, having a predetermined length and width and having two ends, the interior of said tubing having a smooth regular cylindrical shape, having a discharge passageway extending there through.

Cleanliness is very important in any methods used and the equipment must be perfectly sterilized for obvious reasons.

### Syringe:

A syringe is a simple pump consisting of a plunger that fits snuggly into a cylindrical tube, allowing the syringe to take in and expel liquid substances through an orifice located at the opening end of the tube.

Medical-grade disposable syringes and micro liter disposable syringes are often used for measuring and transferring solvents/liquids where high precision is not required.

Cleanliness is very important in any methods used and the equipment must be perfectly sterile for obvious reasons. Preferably, a new syringe should be used every time to provide the cleanliness required. Because syringes are made of inexpensive plastic material and is available on the market at such a low cost, it is feasible to discard the syringe after each use.

### Artificial Insemination:

Artificial insemination is the process by which sperm is placed into the reproductive tract of a female for the purpose of impregnating the female by using means other than sexual intercourse. This is primarily to treat infertility, but is also increasingly used to enable women without a male partner (i.e., single women and lesbians), to produce children by using sperm provided by a sperm donor.

### Intravaginal Insemination:

Intravaginal insemination is a type of artificial insemination that you can use to help increase your chances of pregnancy. Though less common than the other types of artificial insemination, intravaginal insemination is a valuable treatment procedure and is very popular among some patients. Intravaginal insemination places sperm directly inside your vagina in order to increase the chances of fertilization. Intravaginal insemination is usually reserved only for couples facing specific fertility issues. Intravaginal insemination is typically used when a male partner is unable to ejaculate during sexual intercourse, but when the female partner is healthy and fertile. This procedure requires that you ovulate regularly.

### Sexual Partners:

a) Two Female: Lesbian couples are composed of two women. Sex between two females can include frottage, mutual masturbation, and the use of sex toys for vaginal or oral penetration or clitoral stimulation. As with any sexual relationship, people may begin with various forms of foreplay such as fondling, caressing, and kissing, and may or may not experiment with other practices.

b) Male To Female: Heterosexual couples are composed of a male and a female. The adjective heterosexual is used for intimate relationships and/or sexual relations between make and female partners. Heterosexuality refers to “an enduring pattern of or disposition to experience sexual, affectionate, physical or romantic attractions primarily to “persons of the opposite sex”; it also refers to an “individual's sense of personal and social identity based on those attractions, behaviors expressing them, and membership in a community of others who share them.

c) Transgender: Transgender is a general term applied to a variety of individuals, behaviors, and groups involving tendencies that diverge from the normative gender role (woman or man) commonly, but not always, assigned at birth, as well as the role traditionally held by society. Transsexual people identity as, or desire to live and e accepted as, a member of the sex opposite to that assigned at birth. Many transsexual people have a desire to alter their bodies. These physical changes are collectively known as sex reassignment therapy and often include hormone replacement therapy and sex reassignment surgery.

1) Impotency: Erectile dysfunction, oftentimes called “impotence” is the repeated inability to get or keep an erection firm enough for sexual intercourse, or a tendency to sustain only brief erections.

Since an erection requires a precise sequence of events, erectile dysfunction can occur when any of the events are disrupted. The sequence includes nerve impulses of the brain, spinal column, and area around the penis, and response in muscles, fibrous tissues, veins, and arteries in and near the corpora cavernosa. Experts believe that psychological factors such as stress, anxiety, guilt, depression, low self-esteem, and fear of sexual failure, cause 10-20 percent of erectile dysfunction cases. The penis contains two chambers called the corpa cavernosa, which run the length of the organ. A spongy tissue fills the chambers. It contains smooth muscles, fibrous tissues, spaces, veins, and arteries.

An erection begins with sensory or mental stimulation, or both. Pulses from the brain and local nerves cause the muscles of the corpora cavernosa to relax, allowing blood to flow in and fill the spaces. The blood creates pressure in the corpa cavernosa, making the penis expand. The membrane helps trap the blood in the corpora cavernosa, thereby sustaining an erection.

2) Low-sperm count: Low sperm count, a.k.a. oligozgospermia, is one of the main causes of male infertility (<20 million spermatozoa one ml of ejaculate). A reduced amount of ejaculated semen may be caused by a structural abnormality in the tubes transporting the sperm. Genetics, hormonal disorder, testicular injury/disease, malnutrition, overheating (i.e., excessive heat from saunas, hot tubs, etc.), smoking drugs (e.g., cocaine, marijuana); excessive alcohol consumption, prescription, environmental toxins, obesity, stress and cycling; may cause nerves to be damaged, thus causing an assault to the testicles, which may affect sperm production, ultimately leading to a low sperm count and low fertility.

3) Testicular Cancer: Testicular cancer is a cancer that develops in the testicles, a part of the male reproductive system. Testicular cancer is common among makes aged 15-40 years of age. However, males in their mid-twenties are amongst the higher end of those who get testicular cancer. Although testicular cancer has one of the highest cure rates of all cancers, men who suffer testicular cancer often have to undergo extensive radiation therapy and chemotherapy, thus rendering the sperm ineffective. Consequently, heterosexual partners who desire to conceive during the treatment process usually use artificial insemination as an alternative method to natural insemination.

### Cervix:

The cervix is a cylindrical shape that protrudes through the upper anterior vaginal wall. Stimulation of the cervix, for some women, leads to an orgasm. During an orgasm, the cervix convulses and the external outer cervix dilates, which causes the cervix to draw semen in the vagina into the uterus, thus increasing the likelihood of conception. This process is called the “upsuck theory of female orgasm.”

It has been shown scientifically that the cervix, as it constricts, begins to act as a mini vacuum; whereas the cervix sucks the semen (sperm) directly into the opening of the cervix. Because the constriction and suctioning occurs only at the opening of the cervix, using a syringe to inject sperm directly through the dildo and directly into the opening of the cervix as the cervix is constricting, would enable a partner to artificially inseminate their sexual partner.

### Orgasm:

An orgasm is the peak conclusion of the plateau phase or sexual response cycle, and may be experienced by both males and females. An orgasm is typically correlated with the stimulation of the penis in males and with the stimulation of the clitoris in females. Mixed gender or same gender partners can both stimulate and learn to control each other's orgasms.

During an orgasm, a male experiences rapid rhythmic contractions of the anal sphincter, the prostate, and the muscles of the penis. Normally, as a man ages, the amount of semen he ejaculates diminishes, and so does the duration of orgasms. During an orgasm, semen is usually ejaculated and may continue to be ejaculated for a few seconds after the euphoric sensation gradually tapers off.

Female orgasms begin with strong muscle contractions. Theses contractions can be finished within four (4) seconds or last up to about fifteen (15) seconds. Also, the inner two-thirds of the vagina usually open up even more, while the uterus contracts.

Rhythmic muscle contractions occur in the outer third of the vagina, the uterus, and anus. The first muscle contractions are the most intense and occur at a rate of about one (1) per second. As the orgasm continues the contractions become less intense and occur at a more random rate. During female orgasm, a woman's pelvic muscles contract and her cervix repeatedly dips into a pool of semen in the vaginal canal, as if to ensure that sperm would proceed by the external orifice of the uterus, making conception more likely.

### Ovulation:

Ovulation occurs when a mature egg is released from the ovary, pushed down the fallopian tube, and is available to be fertilized. The lining of the uterus thickens to prepare for a fertilized egg. Once a woman is determined to be a suitable candidate for artificial insemination, the next step is pinpointing the time of ovulation. An ovulation test, basal body temperature chart and a cervical mucus examination, may be used.

Ovulation cycle is divided into two parts: the first part of the ovulation cycle is called the Follicular Phase. This first half of the cycle can differ greatly for each woman lasting anywhere from 7-40 dates. The second part of the ovulation cycle is called the Luteal Phase is from the day of ovulation will determine how long your cycle is.

Epstein (U.S. Pat. No. D270,280, Aug. 23, 1983) shows a vibrator attachment with a straight cylindrical member presumably intended for insertion in the vagina or anus. A commercially-available version of this design, sold under the trademark The G Spotter, incorporates a slight crook at the end of this cylindrical member. The external portion of the device can be placed in contact with the clitoris, thus stimulating both areas simultaneously. However, the device does not facilitate direct sexual contact with a partner. Also, the proportions of this device (i.e., short length of the cylindrical member and length and orientation of the crooked end) do no allow thrusting. Moreover, the device lacks the use of a syringe, for movement of fluid material between the syringe and the cylindrical member, for the use of artificial insemination.

Sekulich (U.S. Pat. No. 3,996,930, Dec. 14, 1976) describes a v-shaped vaginal dildo with an external leg, which stimulates the clitoris. However, the internal portion is not intended to be vigorously thrust in and out of the vagina in use. Further, the internal portion does not have cylindrical tubing incorporated for the passage of fluid material from the distal end of the dildo, to the opening of the vagina.

Jacobs (U.S. Pat. No. 5,853,362, Dec. 29, 1998) describes a glandular stimulator device method to be worn internally by a woman that allows her to be stimulated vaginally without the use of straps, buckles, or the like. The stimulators distal end is shape like a dildo to be used for vaginal or anal penetration of a partner. Although the stimulator is used for pseudo genital-to-genital contact, it does not allow the wearer to artificially inseminate their partner because it lacks the use of a dildo with cylindrical tubing, which would allow for semen passage into the vagina of a sexual partner.

Cassou (U.S. Pat. No. 4,261,361, Apr. 14, 1981) describes a syringe mounted to a push-rod for expelling semen for artificially inseminating a domestic animal. Because this invention is mounted to a sheath, the person doing the artificially insemination has the ability to inject semen into the animal without inhibition. Although this is a particularly advantageous use of the embodiment of the invention, it does not allow the use of a harness to hold the syringe, while injecting the semen. It further lacks the use of a dildo, thus eliminating genital-to-genital contact. Lastly, the artificial inseminating process with this device is between a domestic animal and a human, instead of two humans.

Hladky (U.S. Pat. No. 7,241,261, Jul. 10, 2007) describes a dual use syringe containing a tubular body with a straw, that is positioned within a sheath that is attached to a domestic animal for artificial insemination, by penetration the uterus with a syringe or a gun. Although this device uses a syringe that is attached to the body of a domestic animal via a sheath, to push fluid material into the uterus, the sheath is worn by the animal, not the person injecting the fluid. Moreover, the device lacks the use of a dildo, which would enable pseudo genital-to-genital sexual contact between two individuals. Lastly, the device is made for human-to-animal use.

McAllister (U.S. Pat. No. 5,127,396, Jul. 7, 1992) shows a phallus device formed of resilient material in combination with a lug formed of substantially rigid material, attached to a harness, presumably intended for the wearer to sexually penetrate the vagina or anus of their sexual partner by pseudo genital-to-genital contact. However, the device lacks the use of a syringe, which would hold material fluid for the use of artificial insemination, It further lacks the plug (dildo) with cylindrical tubing, which would transport the material fluid from the syringe to the opening of the vagina.

### OBJECTS AND ADVANTAGES

Accordingly, several objects and advantages of the present invention are to provide a sexual device to be worn by a woman, man, or transgender person that will allow said person to artificially inseminate their sexual partner by pseudo natural means. Regardless of the gender of the wearer, the device provides an easy-to-use method for pseudo genital-to-genital sexual contact. The harness allows the wearer to attach a dildo, with cylindrical tubing running horizontally through its center (which said dildo optionally allows said wearer to penetrate their partner's vagina), and a syringe to its entity. This allows said wearer the ability to manipulate the amount of material fluid flowing from the syringe, through the cylindrical tubing, to the head of the dildo and into said wearer's sexual partner's vaginal canal.

Thus, the present invention is unique in simultaneously allowing, in a single device, an intimate way to artificially inseminate one's sexual partner. By placing the various objects listed above into one sexual device, it gives an alternative approach to the non-intimate methods currently used for artificial insemination.

A variety of options is an important factor when it comes to sexual intimacy. The present device provides a novel approach to genital-to-genital sexual contact by enhancing a healthy, intimate sexual relationship between partners wishing to conceive through artificial insemination.

### SUMMARY OF INVENTION

In accordance with the present invention, an Intimate Inseminator comprises a harness device constructed of sufficiently rigid material, so that the frontal lobe of the device is a dildo, with a cylindrical tubing incorporated, whose distal end attaches to a syringe, so that the wearer can manipulate the amount of material fluid flowing from the syringe. The Intimate Inseminator provides an easy-to-use method for pseudo genital-to-genital contact between two female partners, male and female partner, and transgender partners, to artificially inseminate in an intimate way. By placing the four various objects listed above into one sexual device, it gives an alternative approach to the various methods of artificial insemination.

Further objects and advantages of the present device will become transparent from consideration of the drawings and ensuing descriptions.

### BRIEF DESCRIPTION OF THE DRAWINGS

FIG. 1 is a perspective view of a harness

FIG. 2 is a horizontal view of a dildo

FIG. 3 is a perspective view of the inside and outside of a dildo with cylindrical tubing

FIG. 4 is a horizontal view of dildo with cylindrical tubing

FIG. 5 is a horizontal view of a cylindrical tube

FIG. 6 is a horizontal view of a syringe

FIG. 7 is a perspective view of the preferred embodiment of an Intimate Inseminator (right-handed) according to the invention

FIG. 8 is a perspective view of the preferred embodiment of an Intimate Inseminator (left-handed) according to the invention

FIG. 9 is a horizontal view of a sheath (right-handed)

FIG. 10 is a horizontal view of a sheath (left-handed)

FIG. 11 is a horizontal view of boy shorts

FIG. 12 is a horizontal view of lacy women's underwear

### DETAILED DESCRIPTION OF INTIMATE INSEMINATOR

### Preferred Embodiment

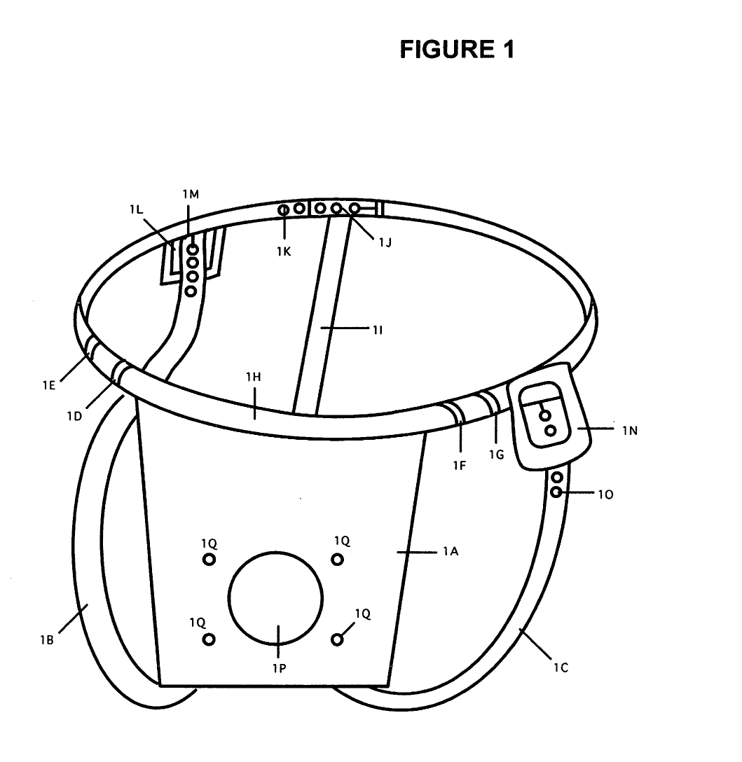

### 4-Strap Harness (FIGS. 1, 7, 8)

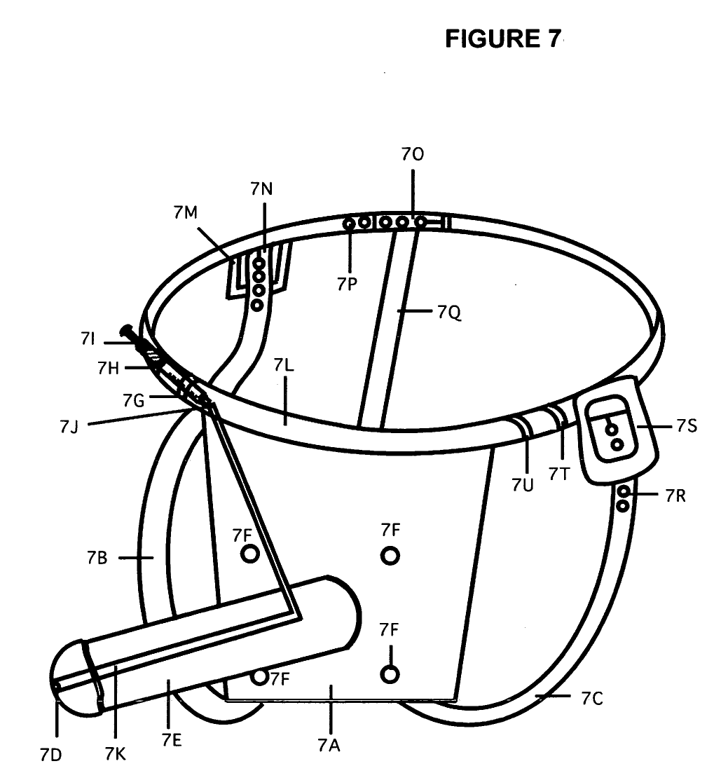

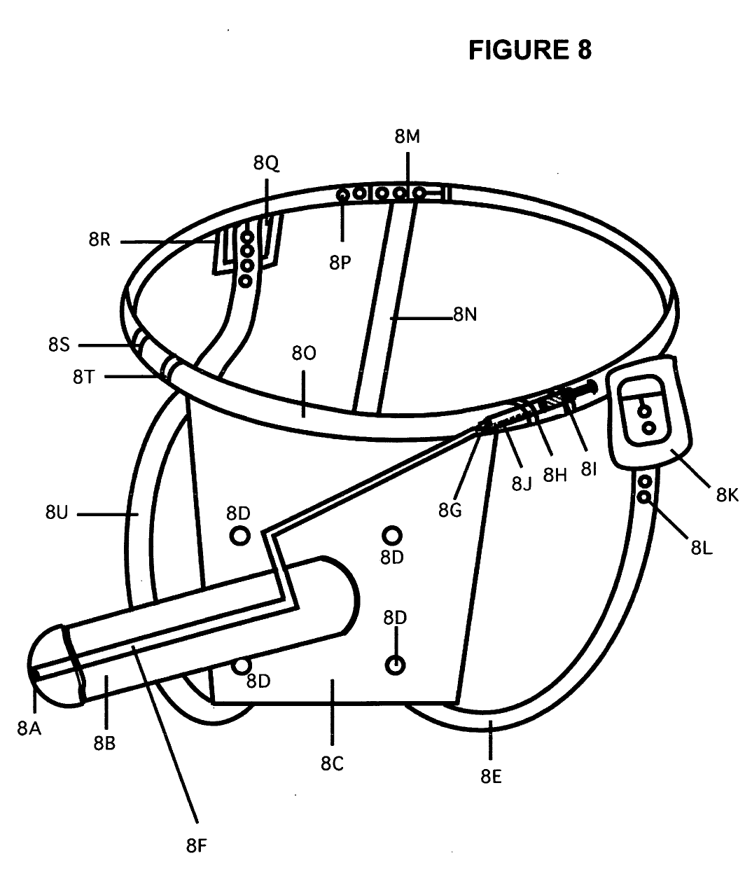

As shown in FIGS. 7 and 8, the preferred embodiment of the invention comprises a harness, dildo, cylindrical tube, and a syringe.

As shown in FIGS. 1, 7, and 8, the harness must be constructed of sufficiently rigid material so that it is sturdy, yet comfortable for the wearer.

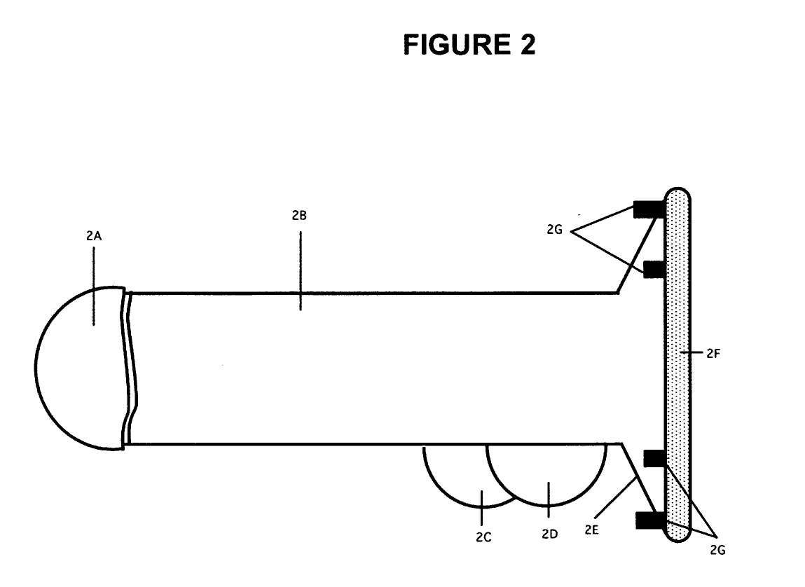

As shown in FIGS. 2, 3 and 4, the dildo should be attached to a rubber backing with four male strap fasteners (2F, 2G, 3H, 3I, 4H, 4I). The size of the dildo may vary between 1.5″-2.5″, and can be constructed of various composite materials (metal, glass, gel, plastic, wood, or rubber). However, silicone is the preferred material.

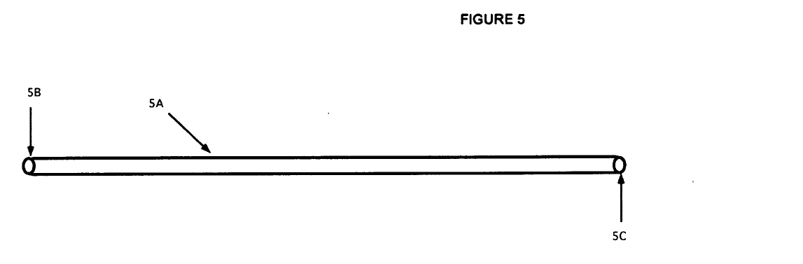

As shown in FIG. 5, the cylindrical tube has an orifice 5B and a distal end 5C. The cylindrical tube should be constructed of clear flexible plastic and should be 100 cm in length.

Cleanliness is very important in any methods used and the equipment must be perfectly sterilized for obvious reasons.

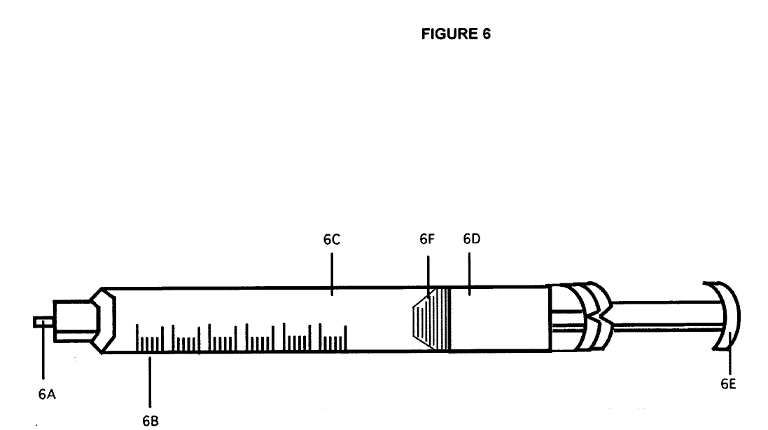

As shown in FIG. 6, the syringe has an orifice 6A, barrel 6C and a plunger 6D. Syringes are usually constructed of clear plastic or glass material and vary in sizes. Our preferred size is 5 ml.

Cleanliness is very important in any methods used and the equipment must be perfectly sterile for obvious reasons. Preferably, a new syringe should be used every time to provide the cleanliness required.

Because syringes are made of inexpensive plastic material and is available on the market at such a low cost, it is feasible to discard the syringe after each use.

Preferred materials for the manufacture of the Intimate Inseminator include: leather, polyurea (silicone), clear flexible plastic tubing, and plastic. Such materials may be purchased from various markets/supply houses, throughout the United States.

The preferred method of manufacture is as follows:

First, a pattern of a harness is made of leather, or any other material that is compatible with leather (i.e., pleather or the like). This pattern has the shape of a 4-strap harness, one that is designed to be sturdy and firm enough to fit the wearer so that said wearer will be able to be comfortable and have control of all attachments (which are preferred to be a dildo/sheath and a 5 ml syringe).

The center waist strap FIGS. 1H, 7L, 80 which is adjustable from 26″-50″ is manufactured to go around the wearer's waist, like a belt. The rear portion of the center waist strap FIGS. 1J, 7O, 8M, will have multiple small circular holes (eyelets) punched 0.5″ apart, in order for the center waist strap to fit snuggly on most body types. The right and left rear sides of the center waist strap will have solid metal buckles.

Adjustable non-slip right and left leg leather straps or any other material that is compatible with leather (i.e., pleather or the like) FIGS. 1B, 1C, 7C, 7B, 8E, 8U are attached to the right and left belt buckle of the center waist strap FIGS. 7N, 7S, 8Q, 8K. Each strap will have multiple small circular holes (eyelets) punched 0.5″ apart FIGS. 1O, 1M, 7N, 7R, 8L, 8Q in order for each leg strap to fit snuggly around the thigh of most body types. Each strap is attached to the bottom edge of the Flange FIGS. 1A, 7A, 8C.

The center core strap FIGS. 1I, 7Q, 8N, is adjustable from 12″-32″ in length with a medium circular hole (eyelet) at the distal end of the strap, begins at the bottom edge of the Flange FIGS. 1A, 7A, 8C and connects to the rear of the waist strap FIGS. 1J, 7O, 8M.

Next, two sets of circular leather straps or any other material that is compatible with leather (i.e., pleather or the like), that are 5.1 cm in diameter, are connected at both the right and left sides of the center waist strap (for the purpose of holding a 5 ml syringe {see FIGS. 6, 1D, 1E, 1F, 1G, 7G, 7H, 7U, 7T, 8S, 8T, 8H, 8I}). Each circular strap will be stitched snuggly enough to hold a 5 ml syringe (FIG. 6) through each loop.

Next, a Flange FIGS. 1A, 7A, 8C is attached to the top of the waist strap and is formed in the shape of a trapezoid, with the top edge being wider in diameter than the bottom edge. A circle shaped hole (size ranging from 1.5″-2.5″ in diameter, depending on the dildo/sheath size) is carved out of the middle-lower center of the Flange. Four female snap fasteners (size ranging from ⅜″- 9/16″) are attached to the Flange, near each corner of the circle shaped hole FIGS. 1Q, 7F, 8D.

Lastly, a dildo FIG. 2 made of silicone or any other composite material (i.e., metal, glass, gel, plastic, wood or rubber) which is in the form of a penis (size of dildo may vary, depending upon users size preference), is adhered to the back of the attachment base (which is comprised of rubber backing and four male fasteners {size ranging from ⅜″- 9/16″}) FIGS. 2F, 2G. Said dildo is then attached to the Flange, via the male-to-female snap fasteners FIGS. 7, 8.

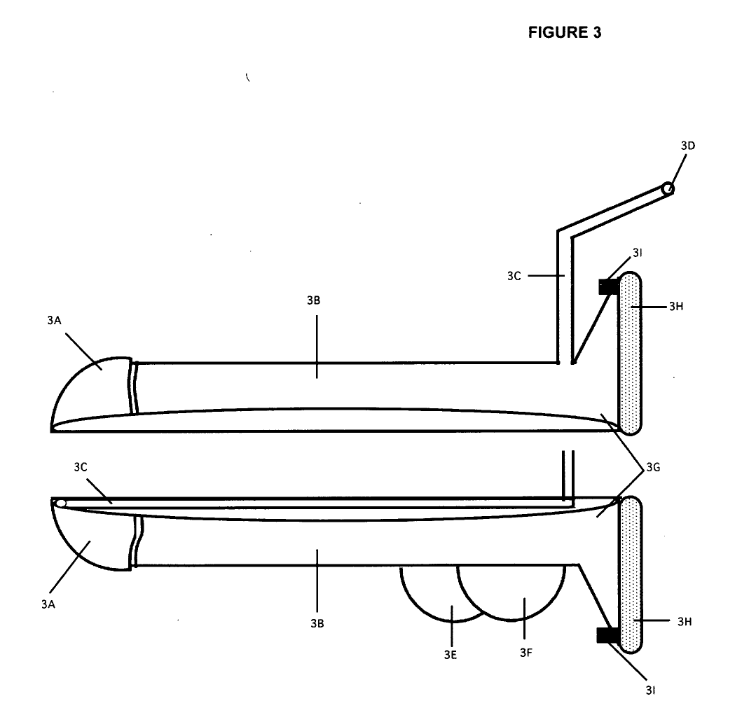

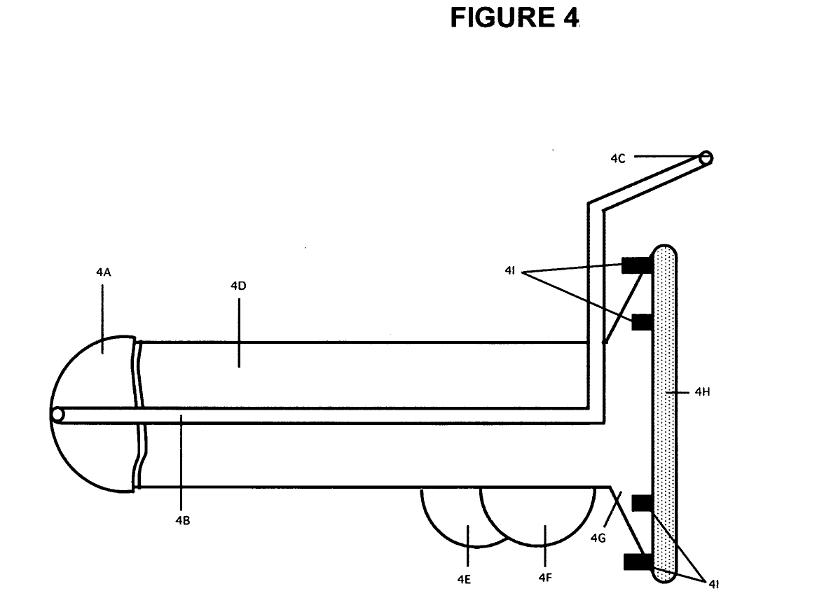

The mold of the dildo (pattern shown in FIGS. 3, 4) will have a cylindrical tube (preferably 100 cm tubing made of clear flexible plastic) FIG. 5 that will begin 0.8 cm behind the orifice (head) of the dildo and will run horizontally through the core of the dildo, curving 90 degrees upward, approximately 5.1 cm before the distal end FIGS. 3C, 3D, 4B, 4C, 7D, 7K, 7J, 8A, 8F, 8G.

To create the dildo with cylindrical tubing, mold-making silicone is poured into two pre-carved bases and is allowed to cure eight hours before removing the two halves from the base.

Once cured, a line approximately 152.4 cm in length and approximately 22 mm in width, curving 90 degrees upward approximately 5.1 cm from distal end, is carved out of both molds FIGS. 3, 4.

The mold is inspected and cleaned. A cylindrical tube is placed in the carved out portion of the bottom half. The mold is carefully closed and bound. A pre-determined quantity of polyurea (or silicone) is poured to slightly overfill the mold. The dildo is de-molded in 24 hours and any holes in the cast are filled with the same material and smoothed over. The finished dildo is allowed to cure for seven days. Manufacture of the alternative embodiments described is similar to that of the preferred embodiment.

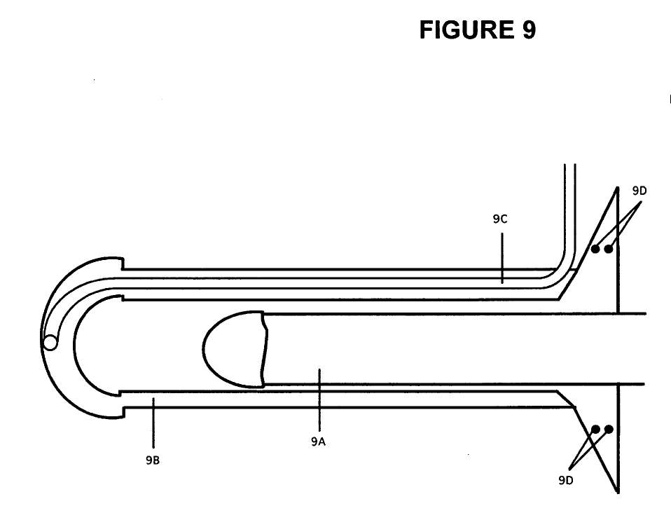

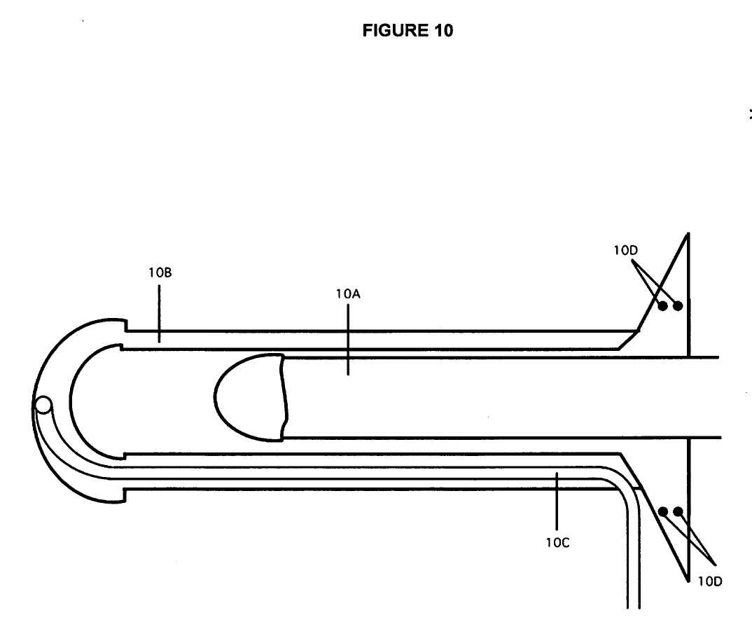

FIGS. 9, 10 illustrate a sheath that can be used as an alternative attachment to the Flange (instead of a dildo, see FIGS. 7E, 8B). In use, the alternative preferred embodiment allows the user to place his penis directly into the sheath.

Like the dildo, the sheath is made of silicone or any other composite material (i.e., metal, glass, gel, plastic, wood or rubber), and has male fasteners FIGS. 9D, 10D. However, unlike the dildo, the sheath does not adhere to a rubber backing.

The mold of the sheath (pattern shown in FIGS. 9, 10) is similar to that of the mold of the dildo, differing mainly by the addition of a hollowed groove in the center of each mold to create an opening large enough for a penis to fit firmly, yet comfortably inside.

The utility of the sheath is as that of any dildo, as it allows the wearer to insert the sheath into their partner's vaginal canal.

### Alternative Embodiment

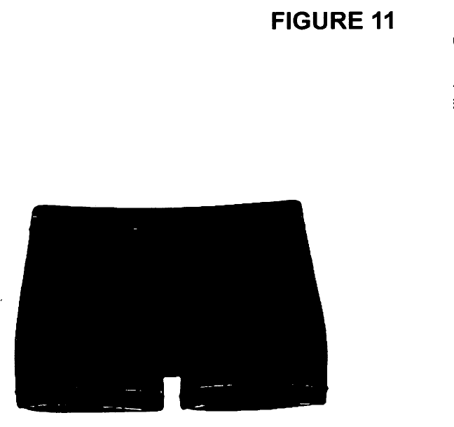

### Boyshorts Harness (FIG. 11)

FIG. 11 depicts an alternative embodiment of the Intimate Inseminator, differing mainly from FIGS. 1, 7, 8 in the material of the harness. Whereas the leather harness is sewn into boyshorts; therefore, the alternative embodiment is described as a Boyshorts Harness.

### Alternative Embodiment

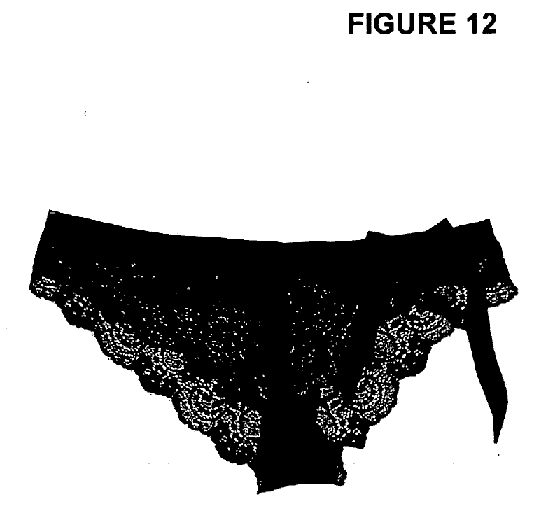

### Lacy Panty Harness (FIG. 12)

FIG. 12 depicts an alternative embodiment of the Intimate Inseminator, differing mainly from FIGS. 1, 7, 8 in the material of the harness. Whereas, the leather harness is sewn into lacy women's underwear; therefore, the alternative embodiment is described as a Lacy Panty harness.

### Alternative Embodiment

### Environmentally Friendly Harness (No Picture Depicted)

The Environmentally Friendly Harness is an alternative embodiment of the Intimate Inseminator, differing mainly from FIGS. 1, 7, 8 in the material of the harness. Whereas, the harness material is made out of organic fibers; therefore, the alternative embodiment is described as an Environmentally Friendly Harness.

### CONCLUSIONS, RAMIFICATIONS, AND SCOPE

Thus the reader will see that our purpose for creating The Intimate Inseminator is to enable both same sex, mixed sex partners, and transgender partners to use the dildo as a means of getting the head of the dildo as close to the opening of the cervix as possible, while using the attached syringe and tubing to inject the sperm directly through the dildo into the opening of the cervix. Our goal is to increase a woman's likelihood of pregnancy, via pseudo genital-to-genital contact.

By using a syringe to artificially inseminate a partner (e.g., same sex partners, mixed sex partners, or transgender partners), the process of artificial insemination is impersonal, at best. By using The Intimate Inseminator, both same sex partners and mixed partners have the ability to attempt impregnation, intimately, by using pseudo genital-to-genital contact.

On The Intimate Inseminator the harness will hold both the dildo and syringe in place during sexual intercourse. The harness can be made up of various different styles, to enhance the ambiance or eclectic tastes of the individuals using it. In addition, different styles of dildos (e.g., size, color, material, etc.) can be used with a harness. Lastly, a syringe attached to tubing can be placed on either side of the harness, to accommodate both right and left-handed individuals.

While the above description contains many specificities, these should not be construed as limitations on the scope of the invention, but rather as an exemplification of one preferred embodiment thereof.

Accordingly, the scope of the invention should be determined not by the embodiments illustrated, but by the appended claims and their legal equivalents.

1. A device for allowing a partner to artificially inseminate another partner by use of pseudo natural means comprising:
a harness, generally being made of leather type material but can also be made by using lacy, nylon, metal, elastic and eco-friendly materials;
said materials being constructed of sufficiently rigid material so as to minimize discomfort by said wearer's body when performing sexual activity; with a dildo attached to the frontal lobe of said harness, to provide sturdiness and secure said dildo in place, to make the sexual performance unproblematic.
said dildo beginning and ending having a predetermined length and width of a cylindrical tube within the dildo which is then attached to a predetermined sized syringe;
said dildo being constructed of silicone, metal, glass, gel, plastic, wood, rubber or composite materials to accommodate all types of sexual preferences; Additionally, for men who are impotent, said dildo can be constructed as a hollow dildo (outer sheath/inner sheath and a cylindrical tube within sheath for use as an artificial insemination device/method for the insemination of a sexual partner), so as to accommodate said wearer's sexual dysfunction;
said cylindrical tubing having a predetermined length and diameter, will be constructed to run horizontally through the center of said dildo. The opening of said tubular element will be positioned slightly before the head of said dildo, to eliminate possible damage to the vaginal canal and cervix. Said tubular element will continue through the center of said dildo, protruding past the end of said dildo, with a specified length of tubing remaining so said tubular element can be attached to the head of a syringe, which will be located at either the right or left side of said harness, to accommodate both right and left-handed users;
said syringe further having a predetermined size and configuration of 5 ml so that the semen will flow from said syringe through the cylindrical tubing, to the head of said dildo, into the sub-cavity of a woman's vagina; thus allowing as much semen as possible to flow into the cervix for maximum probability of fertilization.

2. The device of claim 1 wherein said harness generally being made of leather type material, but can also be made by using lacy, nylon, metal, elastic and eco-friendly materials, will be proportionately adapted to the wearer.

3. The device of claim 1 wherein said dildo is used as an apparatus to cause sexual stimulation and artificial insemination of said wearer's sexual partner.

4. The device of claim 3 wherein material comprises: material selected from the group consisting of silicone, metal, glass, gel, plastic, wood, rubber or composite materials.

5. The device of claim 1 said tubular element is of a length and diameter adapted to ensure maximum flow of the spermatozoa from said wearer's syringe when hooked from the harness to the exterior (distal end) of the tubular element so that the spermatozoa will flow to the head of said dildo and whereby said wearer may artificially inseminate their sexual partner by using said method.

6. The device of claim 1 wherein said syringe having a predetermined size and configuration of 5 ml to ensure maximum flow of the spermatozoa through the said tubular element whereby said wearer can artificially inseminate their sexual partner by using a single thrust of the syringe, which is located at the right or left of the harness to ensure maximum probability of fertilization.

7. A method for artificially inseminating one's partner by pseudo natural means, comprising:
a) emplacing a dildo with cylindrical tubing constructed through the center of said dildo, which is attached to a harness constructed of sufficiently rigid material, said harness having a syringe attached to either the right or left side of said harness being of a size so that the flow of spermatozoa easily maneuvers from the end of syringe through cylindrical tubing to the head of said dildo, to fill the vaginal cavity of said wearer's partner with spermatozoa to increase probability of insemination, and
b) said dildo can also be constructed as a hollow dildo (outer sheath and inner sheath) so as to accommodate a transgender and/or a male experiencing sexual dysfunction issues (i.e., impotency, low sperm count, or the like).

8. The method of claim 7 wherein said dildo being constructed of silicone, metal, glass, gel, plastic, wood, rubber and composite materials to accommodate all types of sexual preferences, and being of length and proportion sufficient to accommodate said wearer's and said wearer's sexual partner.

9. The method of claim 7 wherein said material of said harness comprises: material selected from the group consisting of leather, lacy, nylon, metal, elastic and eco-friendly materials, to provide sturdiness and secure said dildo in place, to make the sexual performance unproblematic, all-the-while giving the feeling of genital-to-genital intercourse between said wearer and said wearer's partner.

10. The method of claim 7 wherein said syringe having a predetermined size and configuration of 5 ml to accommodate the specific cylindrical tubing, which will hold the spermatozoa for the artificial insemination process.

11. The method of claim 7 wherein said cylindrical tubing having predetermined length and diameter will be constructed to flow through the center of said dildo thus allowing as much semen as possible to flow horizontally through said cylindrical tubing into the cervix for maximum probability of fertilization.

12. A device for artificially inseminating one's sexual partner by pseudo natural means, comprising:
a) a sexual harness, said harness constructed of sufficiently rigid material so as to minimize discomfort by said wearer's body when performing sexual activity. The frontal lobe of said harness is a dildo with a predetermined size and length to fit comfortably into the sub cavity of a vagina, and
b) a cylindrical tube, said tubing being of predetermined length and width is constructed to begin in the interior lining at the head of said dildo and will continue through to the end of said dildo, said tubing being of length sufficient to extend from distal end of said dildo, is attached to the tip of a syringe, and
c) said syringe being of predetermined size is attached to the right or left side of said harness, and
d) the wearer of said harness can manually inject sperm directly through said syringe, into said cylindrical tubing, through the head of said dildo, being of length sufficient to fit comfortably into the sub cavity of a vagina, whereby the distal end of said dildo rhythmically makes pseudo genital-to-genital sexual contact with said wearer and said wearer's sexual partner, to create an intimate of artificial insemination experience.

13. A device of claim 11 wherein said material comprises: material selected from the group consisting of leather type material but can also be made by using lacy, nylon, metal, elastic and eco-friendly materials.
